(19) United States
(12) Patent Application Publication
(10) Pub. No.: US 2019/0240413 A1
(43) Pub. Date: Aug. 8, 2019

(54) Portable Devices And Methods For Detecting, Identifying And Quantifying Amounts Of Subcutaneously Injected Compounds

(71) Applicant: NGageIT Digital Health, Inc., Toronto (CA)

(72) Inventor: Raj Reddy, Burlington (CA)

(21) Appl. No.: 16/269,249

(22) Filed: Feb. 6, 2019

Related U.S. Application Data
(60) Provisional application No. 62/626,914, filed on Feb. 6, 2018.

(51) Int. Cl. A61M 5/31; A61M 5/142; A61B 5/00

(52) U.S. Cl. CPC A61M 5/31; A61M 39/0208; A61B 5/0064; A61M 5/14244

(57) ABSTRACT

This disclosure relates to portable devices and methods for detecting, identifying and quantifying subcutaneously injected compounds in a subject. In an example, an injector is disposed in a housing, a cartridge is attached to the injector which includes the compound and one or more marker molecules, a near infrared radiation source is disposed in the housing, a monochromator is disposed in the housing through which the near infrared radiation is focused in a narrow bandwidth, a detector is disposed in the housing which includes detects near infrared radiation data absorbed by the marker molecule, a gyroscope identifies the angle of injection, a communication apparatus is disposed in the housing connected to the detector which electrically transmits the data collected by the detector and the gyroscope, and a battery is disposed in the housing connected to the communication apparatus, near infrared radiation source and the detector.

This application claims priority under 35 U.S.C. § 119 (e) from U.S. Provisional Application Ser. No. 62/626,914, filed Feb. 6, 2018 which is hereby incorporated by reference in its entirety.

### TECHNICAL FIELD

This disclosure relates to portable devices and methods for detecting, identifying and quantifying subcutaneously injected compounds in a subject. In some embodiments, the devices and methods described herein detect and identify subcutaneously injected compounds in real time.

### BACKGROUND

Subcutaneously injection of pharmaceutical compounds, such as, for example, insulin, Repatha, Rituxan Hycela, Stelera, CD38, Adulimumab, Rituximab, Trastuzmab, etc. is an increasing important method of drug delivery (see, e.g., Wright et al., Medical Research Archives, vol 5, December 2017; Viola et al., J. Control Release 2018, 286). Subcutaneous delivery of biological molecules avoids inconvenient and expensive intravenous injection which typically requires administration in a hospital setting by skilled personnel. About 50% of the cost of many biopharmaceuticals is associated with delivery of the medicine to the subject.

An important issue with subcutaneous delivery of compounds in a non-hospital setting is compliance with pharmaceutical prescriptions and delivery of the correct dosage. Frequently, patients due to memory loss or simple forgetfulness, fail to inject prescribed medication in a timely fashion or at all, which can lead to serious medical issues. Even if patients comply with self-administration routines in a timely fashion, poor technique in subcutaneous delivery may result in delivery of incorrect dosages, Furthermore, health care professionals, who treat such patients are not aware of the lack of compliance, which may prevent proper remedial action. No methods currently exist for measuring patient compliance with subcutaneous injections directly in real time.

Accordingly, there exists a need for automated portable devices and methods for directly detecting, identifying and quantifying subcutaneously injected compounds in a subject in real time with concurrent reporting to remote users, such, for example, health care professionals. Such devices and methods would be of significant value in measuring patient compliance with prescribed pharmaceutical administration and dosage regimens, thus ameliorating medical issues associated with the failure of subjects to ingest prescribed pharmaceuticals with the correct dosage in a timely fashion.

### SUMMARY

The embodiments disclosed herein satisfies these and other needs by providing portable devices and methods for detecting, identifying and quantifying subcutaneously injected compounds in a subject. In some embodiments, the devices and methods described herein detect and identify subcutaneously injected compounds in real time.

In one aspect, a portable device for detecting, identifying and quantifying a compound subcutaneously injected in a subject is provided. The device includes an injector disposed in a housing, a cartridge attached to the injector which includes the compound and one or more marker molecules, a near infrared radiation source disposed in the housing, a monochromator disposed in the housing through which the near infrared radiation is focused in a narrow bandwidth, a detector disposed in the housing which detects near infrared radiation data absorbed by the marker molecule, a gyroscope, which identifies the angle of injection, a communication apparatus disposed in the housing connected to the detector which electrically transmits the data collected by the detector; and a battery disposed in the housing connected to the communication apparatus, near infrared radiation source and the detector.

In a second aspect, a method for detecting, identifying and quantifying a subcutaneously injected compound in a subject is provided which includes the steps of subcutaneously injecting the compound and one or more marker molecules into the subject at an injection angle measure by the gyroscope, irradiating the injected mixture of compound and marker molecule with near infrared radiation provided by a near infrared radiation source, which has been focused with a monochromator, measuring the radiation emitted by the injected marker molecule with a detector, communicating the radiation data collected by the detector via a communication apparatus to a processing apparatus and processing the communicated data to detect, identify and quantify the one or more maker compounds.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates the penetration of skin by various wavelengths of light which include blue, red and infrared.

FIG. 2 illustrates an external view of a portable device described herein

FIG. 3 illustrates a process of identifying and detecting one or more subcutaneously injected compounds in a subject with a portable device described herein.

FIG. 4 illustrates a cross sectional view of a portable device described herein which identifies and detects one or more subcutaneously injected compounds in a subject.

FIG. 5 illustrates the bottom portion of a portable device which identifies and detects subcutaneously injected compounds in a subject.

FIG. 6 illustrates a neural network which may be used in a processing apparatus to analyze data provide by the detector.

### DETAILED DESCRIPTION

Disclosed herein are portable devices and methods for detecting and identifying subcutaneously injected compounds in a subject. Also disclosed herein are marker molecules which may be used to identify the subcutaneously injected compounds.

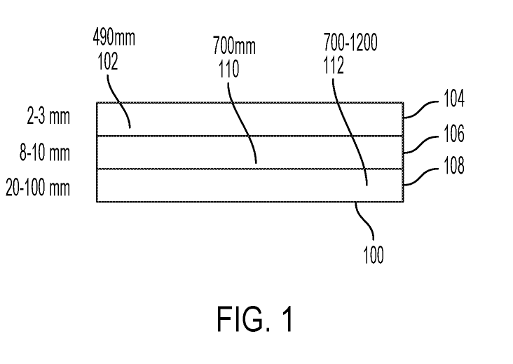

Referring to FIG. 1, the penetration of various radiation wavelengths through the skin is illustrated. Layers 104, 106 and 108 represent the epidermis, dermis and subcutaneous layers. Blue light 102 (λ of about 490 nm) penetrates about 2-3 mm below the skin through the epidermal layer 104 and a slight amount of the dermal layer. Red light 110 (λ of about 700 nm) penetrates about 8-10 mm below the skin to reach the dermal layer 106. Near infrared radiation (λ of about 700-1200 nm) penetrates about 20-100 mm below the skin to reach the subcutaneous layer 108. Accordingly, subcutaneously injected substances are best detected by near infrared irradiation.

However, most pharmaceutical compounds absorb poorly in the near infrared region of the spectrum. Accordingly, a marker molecule which absorbs in the near infrared must be mixed with the compound, which is to be subcutaneously injected. Briefly, the marker molecule must possess several properties in order to be useful in the devices and methods described herein. First, any marker molecule must absorb and emit significant radiation in the near infrared. Second, any marker molecule must be water soluble. Finally the marker molecule must be biocompatible and meet FDA safety standards.

Ideally, marker molecules can be used in isolation. In certain circumstances, marker molecules may be displayed on nanoparticles which may enhance the absorption cross section in the near infrared region. In some embodiments, the nanoparticle is chitosan and a polymer, polyvinyl alcohol nanoparticles or polyvinylpyrrolidine nanoparticles, which may be made by methods well known in the art. In other embodiments, the polymer used with chitosan is tripolyphosphate, HPMC, HPC, PVP, ethyl cellulose, PEG, cellulose acetate phthalate and derivatives thereof, bioadhesive coatings such as, for example, poly(butadiene-maleic anhydride-co-L-DOPA) (PBMAD), etc. In still other embodiments, marker molecules are polypyrrole, polypyrrole-methylene blue composite, pthalocyanines, naphthalocyanines, polymethine, quinones, metal complexes or combinations thereof. The marker molecules may be used with any compatible nanoparticle such as those mentioned above.

Finally, it should be noted that detection of marker molecules may be used to quantify the amount of compound subcutaneously injected in a subject. If known mixtures of compound and marker molecule are subcutaneously injected in a subject, the amount of marker molecule detected by the device can be correlated with the amount of compound subcutaneously injected in a subject. The above may be used to estimate the dosage injected and identify operator problems with subcutaneous injection, including, for example, incorrect angle of injection or incomplete injection.

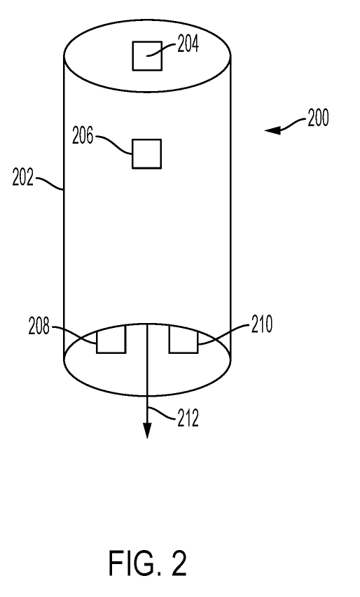

Referring to FIG. 2, illustrated is an example of a portable device 200, which may be used to detect, identify and quantify subcutaneously injected compounds in a subject. Device 200 has a housing 202 which includes power button 204, indicator light 206 which shows the state of the system, portal for near infrared light emission 208, injector 212 and portal for detector 210. As is obvious to those of skill in the art, many other designs and configurations of such a portable device are possible and the above illustration is in no way limiting.

Briefly, a portable device, such as the one illustrated in FIG. 2 collects near infrared spectral data about the presence, identity and quantity of compounds subcutaneously injected compounds in a subject with a detector disposed in the housing. Usually, a marker compound is detected. The detector transmits converts the collected data to a signal (e.g., optical, thermal, electrical, etc.), and transmits the data via a communication apparatus to a processing apparatus which analyzes the data to provide information about the presence, identity and quantity of compounds subcutaneously injected compounds in a subject. The processing apparatus may include, for example, a neural network, which processes the received signals to provide information about the presence, identity and quantity of the compounds subcutaneously injected in a subject and which may transmit the presence and identity of the compounds to a display.

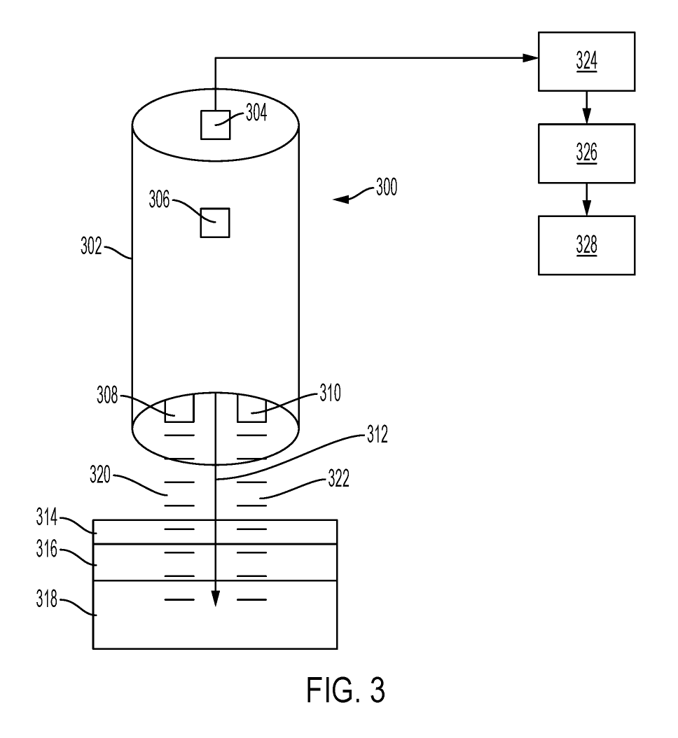

The process described above is illustrated in detail in FIG. 3. Device 300 has a housing 302 which includes power button 304, indicator light 306 which shows the state of the system, portal for near infrared light emission 308, injector 312 and portal for detector 310. Device 312 is emitting near infrared radiation 320 through epidermal layer 314, dermal layer 316 to subcutaneous layer 316, where a compound (not shown) and a maker molecule (not shown) have been injected. The marker molecule, after absorbing near infrared radiation emits radiation 322, which passes through subcutaneous layer 316, dermal layer 316, epidermal layer 314 and portal for detector 310 and is collected by the detector (not shown). The detector (not shown) sends the data to a communication apparatus (not shown), which transmits the data (e.g., by wireless or electrical means) for example, to the cloud 324. The data may be stored in the cloud 324 and may be accessed by processing apparatus 326, which detects and identifies the marker compound and hence the compound subcutaneously injected in the subject and communicates the above result via a display 328 to a user. The communication apparatus may directly transmit data to the processing apparatus 326, thus bypassing cloud 324. Ideally, the process illustrated in FIG. 3 takes place in real time (i.e., at substantially at the same time the event is occurring, with any delay being less than, for example, one minute). However, data may be stored in the cloud before being processed by the processing apparatus and displayed to a user.

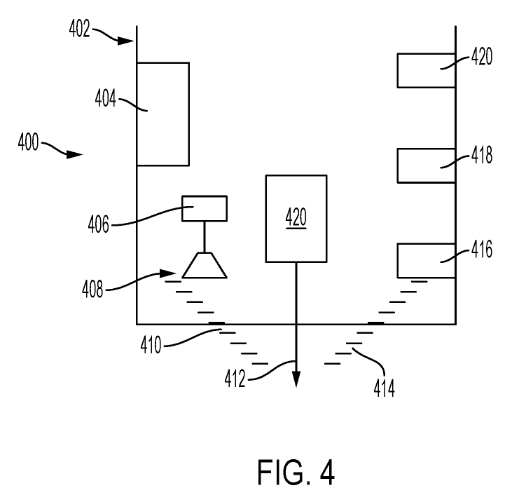

A portable device 400 is illustrated in a sectional view in FIG. 4. Device 400 includes housing 402 which on the left side of the figure contains battery 404, near infrared radiation source 406 and monochromator 408. After passing through monochromator 408, near infrared radiation of a narrow bandwidth 410 is emitted through a transparent portal (not shown). In the center of housing 402 resides an injector 412, to which is attached, for example, a cartridge, which can include the compound and marker molecule which are to be subcutaneously injected. On the right side of housing 402 is shown light 414 emitted by a marker molecule subcutaneously injected in a subject (not shown), which passes through a transparent portal (not shown) to detector 416. Also shown is gyroscope 418 which measures the angle of injection deployed by the user to subcutaneously self-inject compound and marker molecule and communication device 420 which transmits data collected by gyroscope 418 and detector 416 to the cloud or an external processing system. It should be noted that gyroscope 418, detector 416 and radiation source 406 are connected to battery 404 by wiring (not shown). Also not shown is a door on the rear of device 400 which may be used to access housing 402 to replace the injector, cartridge and/or other components as necessary. In some embodiments, the battery is charged by a wireless charger, which is not depicted.

The injector may be a replaceable pen type device such as those used in many insulin pen devices. The cartridge, which contains the compound to be subcutaneously injected and the marker compound is attached to the injector may also be a replaceable cartridge such as those used in insulin devices. Ideally both injector and cartridge are replaced after each use of the device.

Any commercial near infrared radiation source and monochromator may be used in device 400. In some embodiments, LEDs of defined wavelengths are used to emit near infrared radiation.

Commercially available optical sensors are included in detector 416 to collect near infrared radiation emitted by marker molecules. The solid state sensor should be capable of recognizing both incident light and end emitted light. For example, InGaAS photodiode which a range of detection from 850 nm to 1700 nm is an exemplary optical sensor.

Any commercially available gyroscope which is attached to a control board can be used in device 402. An exemplary gyroscope is attached to a PCB control board. The gyroscope measures the angle of injection, which is an important variable in subcutaneous delivery of a pharmaceutical compound. An incorrect angle of delivery can substantially reduce the amount of the pharmaceutical compound that is subcutaneously injected.

An exemplary communication apparatus can be purchased from commercial sources (e.g., Qualcomm® Snapdragon™ SDM845 X20 LTE modem from Qualcomm, Inc. San Diego, Calif.) and is entirely conventional. Many such communication apparati are known in the art and can be used in the portable devices described herein.

An exemplary battery is a lithium ion battery, which are conventional and available from many commercial sources (e.g., Panasonic DMW-BCM14 battery). Many batteries are known in the art and may be used in the portable devices described herein.

The processing apparatus will typically be a conventional general-purpose computer which includes a display device and a communication interface which allows reception and transmittal of information from other devices and systems via any communication interface. The processing module will typically detect and identify the one or more compounds in the breath of the subject by processing the data received from the sensor module with results sent to the display device. Any general purpose computer known in the art which has sufficient processing power to analyze data provided by the detector module may be used in conjunction with the portable devices described herein.

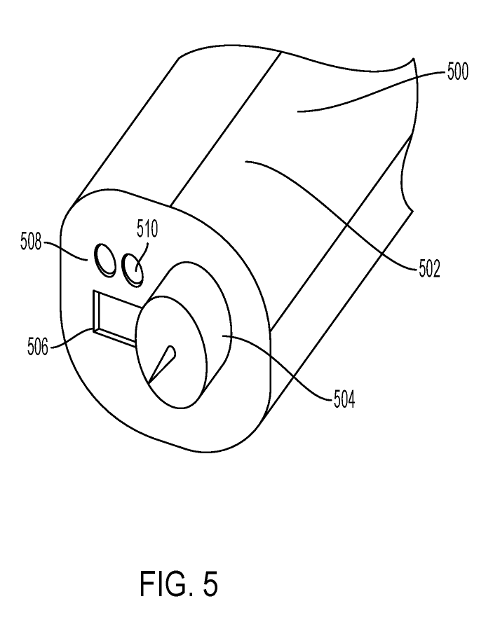

Referring now to FIG. 5, shown are embodiments where two infrared radiation sources are deployed. Device 500 includes housing 502 which includes injector 504, detector portal 506 and two near infrared radiation portals 508 and 510. Primary near infrared radiation source 510 will emit radiation of wavelength of about 1070 which has maximum penetration of the skin. In some situations, some marker molecules may not emit radiation at 1070 or may have identical emittance at 1070. Secondary infrared radiation source 508, which emits radiation at a wavelength of 1670 will assist in unambiguously identifying marker molecules. Relative emission intensity at these two wavelengths will be use in this embodiment to identify marker molecules.

In some embodiments, data from sensors in the sensor module is analyzed using pattern and recognition systems such as, for example, artificial neural networks, which include, for example, multi-layer perception, generalized regression neural network, fuzzy inference systems, etc. and statistical methods such as principal component analysis, partial least squares, multiples linear regression, etc. Artificial neural networks are data processing architectures that use interconnected nodes (i.e., neurons) to map complex input patterns with a complex output pattern. Importantly, neural networks can learn from using various input-output training sets.

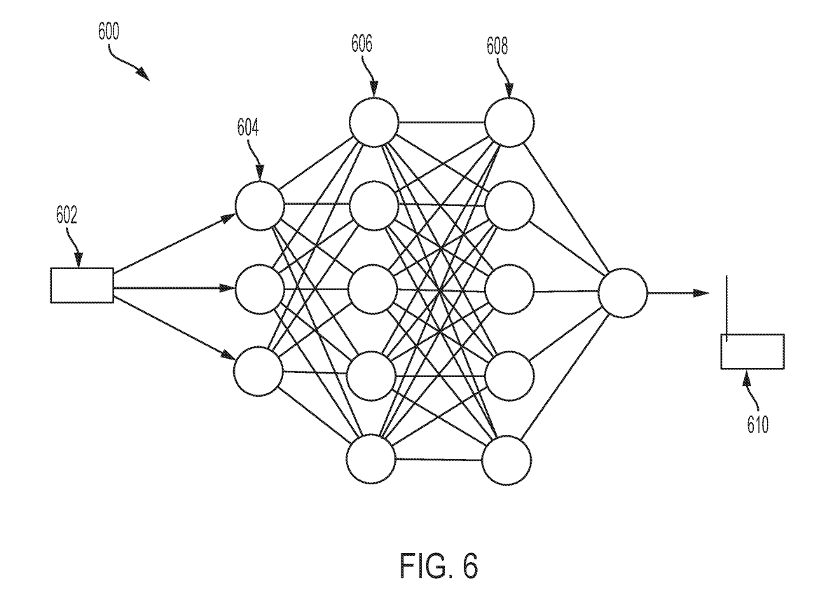

Referring now to FIG. 6, an exemplary artificial neural network 600 which can process data received from the sensor module 602 is illustrated. In general, the neural network uses three different layers of neurons. The first layer is input layer 604, which receives data from sensor module 602, the second layer in hidden layer 606 while the third layer is output layer 608, which provides the result of the analysis at 610. Note that each neuron in hidden layer 606 is connected to each neuro in input layer 604 and each neuron in output layer 608. In the exemplified neural network, hidden layer 606 processes data received from input layer 604 and provides the result to output layer 608. Although only one hidden layer is illustrated in FIG. 6, any number of hidden layers may be used, with the number of neurons limited only by processing power and memory of the general purpose computer. The inputs to the input neurons are inputs from the sensors in the sensor module. If, for example, seven sensors are in the sensor module, then the input layer will have seven neurons. In general, the number of output neurons corresponds to the number of compounds that the sensor module is trained to detect and identify. The number of hidden neurons may vary considerably. In some embodiments, the number of hidden neurons is between about 4 to about 10.

Compounds which may be delivered subcutaneously by the device disclosed herein include, but are not limited to, insulin, Repatha, Rituxan Hycela, Stelera, CD38, Adulimumab, anticancer agents (Rituximab, Trastuzmab, bortezomib, omacetaxine, etc., (luteinizing hormone-releasing hormone analogs, cytokines (e.g., aldesleukin/interleukin-2, interferon-alpha, etc.), monoclonal antibodies, fertility drugs (e.g., Lupron, Gonal-F, Follistim, Ganirelix, etc.), Nuelasta, etc. In some embodiments, the device described herein may be used in conjunction with Halozyme Enhanz technology (Halozyme Therapeutics, San Diego, Calif.) to deliver injectable drugs subcutaneously.

It should be noted that the device described herein may be especially useful for subcutaneous delivery of pharmaceuticals to pediatric populations which require smaller doses than adults. Injectors which are pen devices allow for more accurate and better compliance than a syringe and are useful for children who otherwise would require assistance in receiving a dose.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A portable device for detecting, identifying and quantifying a compound subcutaneously injected in a subject comprising:
an injector disposed in a housing;
a cartridge attached to the injector which includes the compound and one or more marker molecules;
a near infrared radiation source disposed in the housing;
a monochromator disposed in the housing through which the near infrared radiation is focused in a narrow bandwidth;
a detector disposed in the housing which includes detects near infrared radiation data absorbed by the marker molecule;
a gyroscope which identifies the angle of injection;
a communication apparatus disposed in the housing connected to the detector which electrically transmits the data collected by the detector and the gyroscope; and
a battery disposed in the housing connected to the communication apparatus, near infrared radiation source and the detector.

2. The device of claim 1, wherein the near infrared radiation source includes two infrared radiation sources.

3. The device of claim 2, wherein one source emits at about 1070 nm and the other source emits at about 1650 nm.

4. The device of claim 1, wherein a processing apparatus which processes the data transmitted by the communication apparatus to detect, identify and quantify the marker compound is electronically or wirelessly connected to the communication apparatus.

5. The device of claim 4, wherein the processing apparatus transmits the identity of the marker compound detected in the sample from the processing apparatus to a display.

6. The device of claim 1, wherein the marker molecules are detected, identified and quantified in real time.

7. The device of claim 1, wherein the marker molecules are polypyrrole, polypyrrole-methylene blue composite, pthalocyanines, naphthalocyanines, polymethine, quinones, metal complexes or combinations thereof.

8. The device of claim 7, wherein the marker molecules are disposed on nanoparticles.

9. The device of claim 1, wherein the molecule is insulin, Repatha, Rituxan Hycela or Stelara.

10. A method for detecting, identifying and quantifying a subcutaneously injected compound in a subject comprising:
subcutaneously injecting the compound and one or more marker molecules in the subject at an injection angle measure by the gyroscope;
irradiating the injected mixture of compound and marker molecule with near infrared radiation provided by a near infrared radiation source, which has been focused with a monochromator;
measuring the radiation emitted by the injected marker molecule with a detector;
communicating the radiation data collected by the detector via a communication apparatus to a processing apparatus; and;
processing the communicated data to detect, identify and quantify the one or more marker compounds.

11. The method of claim 10, wherein the radiation emitted by the marker molecule is related to the concentration of the marker molecule.

12. The method of claim 11, wherein the concentration of the marker molecule is related to the concentration of the subcutaneously injected compound.
